(19) United States
(12) Patent Application Publication
(10) Pub. No.: US 2009/0143687 A1
(43) Pub. Date: Jun. 4, 2009

(54) DEVICE AND METHOD FOR MANAGING, ARCHIVING AND/OR EVALUATING BLOOD PRESSURE DATA

(75) Inventors: Martin Giersiepen, Muenchen (DE); Dieter Wunder, Schotten (DE); Ulrich Heck, Krefeld (DE); Dirk Freund, Kelkheim (DE); Brigitte Harttmann, Niedernhausen (DE); Stefan Hollinger, Kronberg (DE); Gerrit Roenneberg, Darmstadt (DE); Fred Schnak, Kronberg (DE); Frank Kressmann, Eschborn (DE); Paul Wieneke, Rietheim-Weilheim (DE)

Correspondence Address: DARBY & DARBY P.C., P.O. BOX 770, Church Street Station, New York, NY, 10008-0770, US

(73) Assignee: Kaz USA, Inc., Southborough, MA (US)

(21) Appl. No.: 12/276,774

(22) Filed: Nov. 24, 2008

Related U.S. Application Data
(63) Continuation of application No. 11/912,752.

(30) Foreign Application Priority Data
Apr. 28, 2005 (DE) 102005019750.7

(51) Int. Cl. A61B 5/02

(52) U.S. Cl. 600/485

(57) ABSTRACT

A device and method for managing, archiving and/or evaluating blood pressure data of a plurality of patients. The blood pressure data is acquired using blood pressure monitors. The blood pressure data for each patient is separately stored in an individual storage space in a central data server. A network connection including at least one network terminal is used to input the data into the individual storage space and read out the data from the individual storage space. A control device controls access to the storage spaces by comparing a transmitted access code including a unique device identification code for the blood pressure monitor of the patient corresponding to the individual storage space with a unique access code corresponding to the individual storage space.

### CROSS REFERENCE TO RELATED APPLICATIONS

This application is a continuation of U.S. National Phase application No. 11/912,752, filed on Oct. 26, 2007, under 35 U.S.C. §371 of International Application No. PCT/EP2006/002202, filed on Mar. 10, 2006, and claims the benefit of German Patent Application No. 102005019750.7, filed on Apr. 28, 2005, both of which are incorporated herein. The International Application was published in German on Nov. 2, 2006 as WO 2006/114156 A1 under PCT Article 21(2).

### FIELD OF THE INVENTION

The present invention relates to a device for managing, archiving and/or evaluating blood pressure data, acquired using blood pressure monitors, and providing controlled access to the data. for a plurality of patients, comprising a central data server in which the blood pressure data are stored separately for each patient, wherein the data server has a separate storage space for the blood pressure data for each patient, and a network connection via which data can be input into the respective storage space and can be read out from the respective storage space using various network terminals, and further comprising an access control device for controlling access to the storage spaces, wherein said access control device compares an access code that is transmitted when an attempt at access is made with an access code that is unique to the respective storage space. The invention further relates to a method for managing, archiving and/or evaluating blood pressure data for a plurality of patients, in which blood pressure data for different patients, acquired using different blood pressure monitors, are transmitted via remote data transmission from network terminals to a central data server, where they are stored in storage spaces that are unique to the individual patient, wherein, when an attempt is made to access the stored blood pressure data of a specific patient, as a part of the access attempt an access control device, which is connected to the data server, demands an access code and compares it with an access code that is unique to the respective storage space.

### BACKGROUND

Various ways have been proposed for transmitting patient data, acquired for a patient using suitable measuring instruments, via remote data transmission to a central data server, where they are managed and evaluated. For instance, WO 01/93756 A2 describes a tele-monitoring system, with which an EKG device is used to acquire corresponding patient data on the patient, which are initially stored in the EKG device on a smart card. At the appropriate time, the data are downloaded from the smart card by a card reader device and are transmitted via remote data transmission to a central monitoring station, where they can be presented, analyzed and managed to allow medical diagnosis. Further, DE 202 14 189 U1 describes a device for the mobile measurement and transmission of values for bodily functions, in which the corresponding measuring instrument has a wireless interface for transmitting the measured bodily function values to a mobile telephone, which in turn transmits these data via the mobile radio network to a central evaluation unit.

Using systems like these, which manage the patient data centrally, stress caused by multiple examinations to establish various diagnoses can be prevented, because the data from different examinations can be compiled and made available multiple times. In addition, high quality analytical tools for data analysis and diagnostic support can be used, which for individual users would be unprofitable and too expensive. Nevertheless, such systems leave some room for improvement, especially with regard to accessing the data inventory and the functions offered by the central data server.

### SUMMARY

The present invention provides a device for managing, archiving and/or evaluating blood pressure data, acquired using blood pressure monitors, for a plurality of patients, comprising a central data server in which the blood pressure data are stored separately for each patient, wherein the data server has a separate storage space for the blood pressure data for each patient, and a network connection via which data can be input into the respective storage space and can be read out from the respective storage space using various network terminals, and further comprising an access control device for controlling access to the storage spaces, wherein said access control device compares an access code that is transmitted when an attempt at access is made with an access code that is unique to the respective storage space. The invention further relates to a method for managing, archiving and/or evaluating blood pressure data for a plurality of patients, in which blood pressure data for different patients, acquired using different blood pressure monitors, are transmitted via remote data transmission from network terminals to a central data server, where they are stored in storage spaces that are unique to the individual patient, wherein, when an attempt is made to access the stored blood pressure data of a specific patient, as a part of the access attempt an access control device, which is connected to the data server, demands an access code and compares it with an access code that is unique to the respective storage space

In accordance with the invention, a unique device identification code for the blood pressure monitor of a respective patient can be retrieved and/or provided as an access code for a respective storage space in which the blood pressure data of a respective patient or evaluations derived from said data are stored. The access code that is transmitted when an access attempt is made may be compared with this unique device identification code for the blood pressure monitor. The invention is therefore based upon the idea that each blood pressure monitor bears a unique serial number, which is also stored in manufacturing archives and can therefore be made available, for example, by connecting the manufacturing archives to the central data server. Each patient can thus individually access the central data server at any network terminal, identify himself by the serial number of his device, and query, network or even personally manage his/her alotted storage space. Within this framework it is also possible to program a patient-specific password for the respective storage space into the access control device, which is equipped with the associated appropriate programming. Such a patient-specific password can also be implemented, in addition to the aforementioned access control, with the unique device identification code. For example, such patient-specific passwords can be used as so-called sub-passwords, to which only sub-sections of the respective storage space are allocated. In this way, individual storage sub-spaces can easily be assigned to a plurality of users of a blood pressure monitor, for example within a family. Optionally, the patient-specific password can also be programmed in place of the unique device identification code. In this case, the unique device identification code forms only the initial access control, which is established in advance and can be changed the first time access is gained.

Using the individual device identification code of the respective blood pressure monitor to control access is advantageous because the respective storage space of the data server and/or the associated function blocks can be correctly pre-adjusted or adapted to the data format of the respective blood pressure monitor. From the unique device identification code, the data server can tell what type of blood pressure monitor will be providing the data to be input, allowing the server to be adapted to the data quantity and the data format for the respective patient. This simplifies the configuration of both the data server and the individual storage space.

In one embodiment blood pressure monitors having insertable chip cards may be used, in which case the code for accessing the respective storage space in the central data server can contain a chip card identification code for the respective chip card with which the blood pressure monitor for the respective patient can be equipped. This chip card identification code can be the aforementioned unique device identification code. The chip card identification code can be retrieved or provided in addition to the device identification code as a sub-code, in order to split access to the respective storage space based upon the specific chip card. Frequently, blood pressure monitors are used by a plurality of patients, for example within a household, in which case each patient inserts his/her chip into the device when using it. In this manner, patient-specific access can be ensured in a simple manner.

In principle, the central data server can serve various functions. Archiving and management of the respective blood pressure data on the central data server can be provided so that measurement data acquired previously can optionally be accessed at a much later time. In one embodiment, the data server can also perform an evaluation of the data using appropriate evaluation and analysis tools, and can make the corresponding evaluation data available. The provision of this type of central evaluation unit permits a substantially higher-quality evaluation and analysis of the blood pressure data than would be possible using the blood pressure monitor itself, or even in traditional medical practices, because complex and accordingly costly evaluation programs generally are not economical as an accessory for a single blood pressure monitor, or even for smaller medical practices. Using this type of evaluation unit, a fee charging machine can be provided, which reduces a balance in a fee storage device corresponding to the respective storage space each time the evaluation unit is accessed and/or each time an evaluation is prepared by the unit. Rather than purchasing a costly evaluation program and/or the corresponding hardware, the centrally located evaluation unit can be accessed singularly, with only a relatively small fee being charged for each access. The charging of fees need not be limited to the evaluation unit and/or the evaluations prepared by it. Access to the actual archived blood pressure data can also be subject to fees.

The fee storage device can be provided in a different place in the system. According to one embodiment of the invention, the fee storage device is provided on a chip card, which can be assigned to the blood pressure monitor used to acquire the blood pressure data of the respective patient. When an attempt is made to access the central data server with the chip card, the fee charging device communicates via a chip card reading/writing device, and reduces the amount in the fee storage device accordingly. With this embodiment, an identification and/or assignment of the fee storage device to the respective storage space on the data server is superfluous. The chip card serves as a so-called prepaid card with which the central data server can be accessed as long as the card contains a positive balance. The positive balance on the fee storage device can optionally be reloadable.

In another embodiment, the fee storage device can be provided in the central data server, the fee charging device and/or an associated data storage device. In order to ensure that the amount is deducted from the correct, assigned fee storage device when an attempt is made to access the central data server and its data inventory, the fee storage device can be equipped with an identification code. The identification code can contain a device identification code for the blood pressure monitor of the respective patient and/or a chip card code for a chip card that is assigned to the blood pressure monitor of the respective patient. The fee charging device may identify the respective fee storage device using the device identification code and/or chip card code that is transmitted when an attempt is made to access the central data server.

The transmission of data from the blood pressure monitor to the central data server can be accomplished in various ways. In one embodiment, the measured data can be input into the network terminal by hand, and to transmit the data from there to the central data server. For example, the user can transmit the memory data and measurement times read out on the blood pressure monitor to the central data server.

Alternatively, the data acquired by the blood pressure monitor can be read out of the blood pressure monitor by a reading device. Specifically, the data stored on a chip card in the blood pressure monitor can be read out by a chip card reading device, and then transmitted to the central data server. The chip card reading device may be configured such that it automatically reads a chip card identification code and/or stores it for retrieval. In one embodiment, the reading device can be connected to the network terminal and/or can optionally communicate through a wireless connection via a suitable interface.

In one embodiment, the blood pressure data sets transmitted to the central data server are archived there and can be read out at any time, worldwide, especially via an Internet terminal. In the data server, various evaluations and analyses can be performed and corresponding evaluation and analytical data can be stored.

Additionally, an evaluation of the course over time of the blood pressure data stored in the storage spaces of the central data server can be implemented by suitable evaluation means. Further, a graphic representation of the course of blood pressure and/or pulse over time may be prepared.

Alternatively or additionally, trend analyses can be performed at certain measurement periods, and corresponding trend data can especially be graphically supplied. For example, a tracking of morning measurements over a one-month period can be performed.

Alternatively or additionally, the blood pressure data analysis performed in the central data server can encompass a determination of the average values and standard deviations of all blood pressure data over a specific time period for a specific storage space, in other words for a specific patient.

Alternatively or additionally, daily profiles of pressure levels for a patient can be calculated from the stored blood pressure data using appropriate evaluation means.

In another embodiment of the invention, the system can comprise a data blocking device with which a user can block certain data. The blocking device can preferably be activated with a code and/or password protection.

In order to give a physician, a medical practice or a hospital access to the necessary data on a patient, the access control device can include a master access, with which data from various storage spaces can be accessed. Preferably, this master access can be enabled with a master code and/or a master password.

### BRIEF DESCRIPTION OF THE DRAWINGS

These and additional features will be apparent by referring to the drawings, in which:

FIG. 1: a schematic representation of a device for managing, archiving and evaluating blood pressure data in a central data server according to an embodiment of the invention.

### DETAILED DESCRIPTION

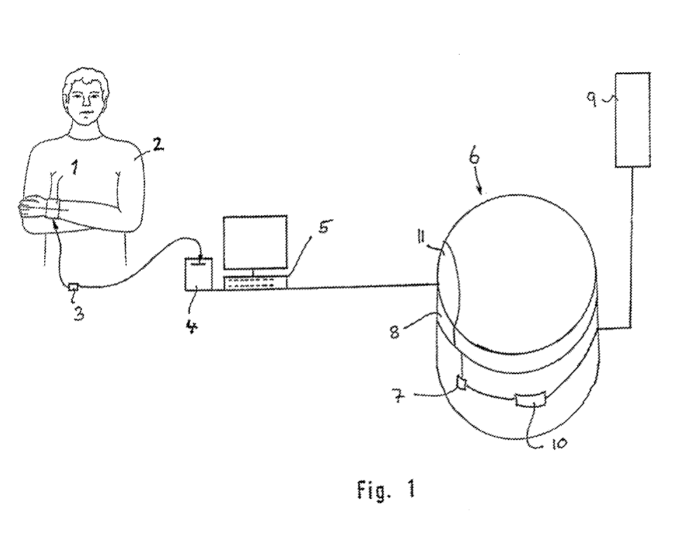

FIG. 1 shows blood pressure of a respective patient 2 being measured daily, optionally over a plurality of measurement cycles, over an extended time period, using a blood pressure monitor 1. The corresponding blood pressure data are first stored in the memory of the blood pressure monitor 1. In this case, the blood pressure monitor 1 advantageously includes a chip card 3 in the form of a memory card or a smart card, which supplies sufficient storage space and can be inserted into the blood pressure monitor 1, as an extension. The aforementioned chip card 3 can be removed from the blood pressure monitor 1 and read out by a chip card reading/writing device 4. In addition to the blood pressure data set, a chip card identification code that is unique to the chip card is read out at the same time, and is used to identify the blood pressure data set.

The chip card reading/writing device 4 can communicate via a suitable interface with an Internet terminal 5 in the form of a PC, with which the data read out from the chip card can be transferred to the central data server 6.

In the data server 6, a corresponding storage space 7 is provided for the respective patient 2, which can be determined using the chip card identification code. Access to the respective storage space is controlled by the access control device 8, which is connected to the central data server 6 and compares the access code transmitted from the Internet terminal 5 with the access code that has been stored for the respective storage space 7, especially the chip card identification code. The chip card identification code that is counter-read by the access control device 8 need not necessarily be stored in the central data server 6 itself, and can instead be stored, for example, in a manufacturer's archive 9 that can be connected to the data server 6. In this archive, additional setup data and control parameters that are unique to the respective blood pressure monitor 1 can be linked to the respective chip card identification code.

The blood pressure data transcribed into the respective storage space 7 can be evaluated and analyzed by an evaluation unit 10. Corresponding evaluations and analytical data are then placed by the evaluation unit 10 in the respective storage space 7 or the evaluation data storage space that is linked to said space, so that they can be retrieved via the Internet terminal 5 in an suitable manner following identification by the access control device 8. The retrieval of the evaluations can also be subject to fees. To this end, the access control device 8 can have a fee charging device 11, which decreases the balance in a fee storage device accordingly each time access is gained to the evaluations, and which can be a part of the storage space 7 or can be allocated to said space. The fee storage device can be loadable in a suitable manner. For example, when a new blood pressure monitor is purchased, a corresponding initial positive balance can be stored on it, which can be reloaded by the subscriber to the central data server 6 through corresponding payments.

With the system shown in FIG. 1, a multitude of advantages can be achieved. To begin with, the electronic archiving, evaluation and visualization of the blood pressure data present a significant benefit to patients. A substantially greater storage depth can be achieved than is possible with a blood pressure log that is kept by hand. In addition, the evaluation of data, which is performed centrally by the central data server 6, is a service that generates cost only when it is actually used. Costly evaluation tools for the respective customers can be eliminated. Furthermore, the evaluation of data always utilizes the most up-to-date processes and can be monitored centrally. Although the respective Internet terminal 5 may not have its own evaluation software and/or have such software installed, the patient 2 can use a printer to print out the evaluations.

Further, the capability of communicating with the respective medical practice and/or care person is simplified substantially. The medical practice need not have any special hardware and software. The blood pressure monitor does not need to be brought to the doctor's office. Additionally, the ability of the physician to access the central data server 6 offers the safety of continuous monitoring of treatment, without the patient having to continuously visit the doctor's practice. Of course, the central evaluation unit 10 also permits the use of complex and therefore high-cost evaluation programs, enabling a higher quality interpretation of the data and determination of treatment measures.

We claim:

1. A device for handling blood pressure data of a plurality of patients acquired using blood pressure monitors, said device comprising:
a central data server in which the blood pressure data are separately stored for each of the plurality of patients;
an individual storage space for the blood pressure data of each patient in the central data server;
a network connection operable to input the data into the individual storage space and read out the data from the individual storage space;
an access control device operable to control access to the storage spaces by comparing a transmitted access code including a unique device identification code for the blood pressure monitor used to acquire the blood pressure data of the patient corresponding to the individual storage space that is transmitted when an access attempt is made with an a unique access code corresponding to the individual storage space.

2. The device according to claim 1, wherein the access code includes a chip card identification code for a chip card that is included with the blood pressure monitor of the respective patient.

3. The device according to claim 1, wherein the access code includes a patient-specific password.

4. The device according to claim 1 further comprising an evaluation unit operable to evaluate the blood pressure data sets stored in the individual storage space;
a fee storage device corresponding to the individual storage space; and
a fee charging device operable
to decrease a balance in the fee storage device whenever at least one of an attempt is made to access the evaluation unit and an evaluation is prepared by the evaluation unit.

5. The device according to claim 17, wherein the fee storage device includes an identification code comprising a device identification code for at least one of the blood pressure monitor operable to acquire the blood pressure data on the respective patient and a chip card code for a chip card, included with the blood pressure monitor, and
wherein the fee charging device is operable to identify fee storage device using the stored identification code.

6. The device according to claim 17, wherein the fee storage device is disposed on a chip card included in the blood pressure monitor, and
wherein the fee charging device uses a chip card reading/writing device to reduce the balance of the fee storage device on the chip card.

7. The device according to claim 1 further comprising a chip card reading device operable to read out the data stored on a chip card of a blood pressure monitor, wherein the chip card reading device is configured to automatically read a chip card identification code.

8. The device according to claim 1, further comprising an evaluation unit operable to at least one of evaluate the blood pressure data sets stored in the storage spaces with respect to their course over time and provide graphic representation of temporal trends of blood pressure levels.

9. The device according to claim 1 further comprising an evaluation unit operable to determine a trend in the blood pressure data of a blood pressure data set in an individual storage space.

10. The device according to claim 1 further comprising an evaluation unit operable to determine mean values and standard deviations of all blood pressure data in an individual storage space within a certain period of time.

11. The device according to claim 1 further comprising an evaluation unit operable to calculate daily profiles of pressure levels for a patient from the stored blood pressure data.

12. The device according to claim 1 further comprising a data blocking device operable to block certain data in a storage space, wherein the blocking device is activated with at least one of code and password protection.

13. The device according to claim 1, further comprising master access capability, with which data from various storage spaces is accessible using at least one of a master code and a master password.

14. A method for managing, archiving and/or evaluating blood pressure data for a plurality of patients, the method comprising:
acquiring blood pressure data from different patients acquired using different blood pressure monitors;
transmitting the blood pressure data to a central data server via remote data transmission;
storing the blood pressure data for each patient in a patient-specific storage space;
comparing an access code corresponding to an access attempt of the stored blood pressure data with a unique access code for the patient specific storage space
wherein the access code corresponding to the access attempt of the stored blood pressure data includes at least one unique device identification code for the blood pressure monitor used to acquire the blood pressure data.

15. The method according to claim 14, wherein the blood pressure data sets stored in the storage spaces are evaluated by an evaluation unit (10), and corresponding evaluations can be stored in the data server, wherein when an attempt is made to access the stored evaluations, a fee charging device automatically reduces the balance in a fee storage device, which is maintained for the respective storage space in which the requested evaluation is stored, based upon the number of access attempts and/or the duration of access.

16. The device according to claim 4 further comprising a fee storage device corresponding to the individual storage space.

17. The device according to claim 16 further comprising a fee charging device operable to decrease a balance in the fee storage device whenever at least one of an attempt is made to access the evaluation unit and an evaluation is prepared by the evaluation unit.
